(19) United States
(12) Patent Application Publication
(10) Pub. No.: US 2005/0208134 A1
(43) Pub. Date: Sep. 22, 2005

(54) Biocompatible polymeric beads and use thereof

(76) Inventors: Shlomo Magdassi, Jerusalem (IL); Frigita Kahana, Jerusalem (IL)

Correspondence Address: WINSTON & STRAWN LLP, 1700 K STREET, N.W., WASHINGTON, DC, 20006, US

(21) Appl. No.: 11/067,316

(22) Filed: Feb. 24, 2005

Related U.S. Application Data
(60) Provisional application No. 60/547,083, filed on Feb. 25, 2004.

(51) Int. Cl. A61K 7/42; A61K 9/24

(52) U.S. Cl. 424/472; 424/59

(57) ABSTRACT

The present invention relates to biocompatible polymeric beads and to biocompatible delivery systems comprising same for controlled or sustained release of bioactive molecules. In particular, the invention relates to polymeric beads having a two-phase core and shell structure and to polymeric delivery systems comprising same that provide sustained release of the bioactive compound.

This application claims the benefit of provisional application 60/547,083 filed Feb. 25, 2004, the entire content of which is expressly incorporated herein by reference thereto.

### FIELD OF THE INVENTION

The present invention relates to biocompatible polymeric beads and to biocompatible delivery systems comprising same for controlled or sustained release of bioactive molecules. In particular, the invention relates to polymeric beads having a two-phase core and shell structure and to polymeric delivery systems comprising same that provide sustained release of the bioactive compound.

### BACKGROUND OF THE INVENTION

Delivery systems and devices for controlled and sustained release of bioactive compounds are well known in the art. A variety of methods have been described in the literature, including the physiological modification of absorption or excretion, modification of the solvent, chemical modification of the active molecule, absorption of drug on an insoluble carrier, use of suspensions and implantation pellets. Other methods include mixing a drug with a carrier such as waxes, oils, fats, and soluble polymers, which gradually disintegrate in the physiological environment resulting in release of the drug. Much attention has been directed to the reservoir type of device, i.e., a device in which an active compound is encased within a polymeric container, with or without a solvent or carrier, which allows passage of compound from the reservoir.

Another type of bioactive compound delivery device, specifically for drug delivery, is the monolithic type in which a drug is dispersed in a polymer from which the drug is released by degradation of the polymer and/or by passage of the drug through the polymer. The release kinetics of a drug from a polymeric delivery system are a function of the agent's molecular weight, lipid solubility, and charge as well as the characteristics of the polymer, the percent drug loading, and the characteristics of any matrix coating.

Previous disclosure by one of the inventors of the present invention and co-workers (U.S. patent Application 20020064541) has used two-phase microcapsules for the preparation of therapeutic or cosmetic compositions for topical application. The core of each microcapsule includes at least one active ingredient and is encapsulated within a microcapsular shell, which is comprised of at least one inorganic polymer obtained by a sol-gel process.

Alginate matrices have been well documented as delivery systems for water-soluble bioactive compounds. For example, U.S. Pat. No. 4,695,463 discloses an alginate based chewing gum delivery system and pharmaceutical preparations. Alginate beads have been used for controlled release of various proteins by a variety of delivery systems, including tumor necrosis factor receptor in cation-alginate beads coated with polycations (Wee, S. F, Proceed. Intern. Symp. Control. Rel. Bioact. Mater., 21: 730-31, 1994); transforming growth factor encapsulated in alginate beads (Puolakkainen, P. A. et al., Gastroenterology, 107: 1319-1326, 1994); angiogenic factors entrapped in calcium-alginate beads (Downs, E. C. et al., J. of Cellular Physiology, 152: 422-429, 1992); albumin entrapped in chitosan-alginate microcapsules; (Polk, A. et al., J. Pharmaceutical Sciences, 83(2): 178-185, 1994); chitosan-calcium alginate beads coated with polymers (Okhamafe, A. O. et al., J. Microencapsul., 13(5): 497-508, 1996); hemoglobulin encapsulated with chitosan-calcium alginate beads (Huguet, M. L. et al., J. Applied Polymer Science, 51: 1427-1432, 1994; Huguet, M. L. et al., Process Biochemistry, 31: 745-751, 1996); and interleukin-2 encapsulated in alginate-chitosan microspheres (Liu, L. S. et al., Proceed. Intern. Symp. Control. Rel. Bioact. Mater, 22: 542-543, 1995).

However, the known delivery systems employing alginate gel beads are used mainly for water-soluble compounds such as proteins or peptides. In addition, these systems suffer from lack of any sustained-release effect due to rapid release of the drug from the alginate beads (Liu, L. et al., J. Control. Rel., 43: 65-74, 1997). To avoid such rapid release, a number of the above systems attempt to use polycation polymer coatings (e.g., polylysine, chitosan) to retard the release of the protein. Alginate beads are disclosed for example in Wheatley, M. A. et al. (J. Applied Polymer Science, 43: 2123-2135, 1991) and Wee, S. F. et al. (Controlled Release Society, 22: 566-567, 1995).

Other types of bioactive compound delivery vehicles for fat-soluble compounds are water-in-oil or oil-in-water emulsions. Emulsions are defined as heterogeneous systems of one liquid dispersed in another in the form of droplets usually exceeding 1 μm in diameter. The two liquids are immiscible and chemically non-reactive or slowly reactive. An emulsion is a thermodynamically unstable dispersed system. Instability is a result of the system's tendency to reduce its free energy by separating the dispersed droplets into two liquid phases. Instability of an emulsion during storage is evidenced by creaming, flocculation (reversible aggregation), and/or coalescence (irreversible aggregation).

There is thus an unmet need for biocompatible polymeric delivery systems, which exhibit controlled, local, and preferably sustained release of bioactive compounds.

### SUMMARY OF THE INVENTION

It is an object of the present invention to provide biocompatible polymeric beads having a two-phase core and shell stricture, wherein the beads are suitable for the delivery of water insoluble as well as water-soluble bioactive compounds.

It is another object of the present invention to provide a biocompatible sustained release polymeric delivery system that delivers a stable, effective concentration of bioactive compounds for extended periods ranging from a few hours to a few months.

It is a further object of the present invention to provide a biocompatible polymeric delivery system for sustained release administration of a therapeutic dose of a therapeutic agent to a target site in a subject, wherein the local concentration achieved at the target site is greater than that achieved when the therapeutic agent is administered orally at maximum tolerated oral dose in human subjects, in oral formulations known in the art.

It is yet further object of the present invention to provide a biocompatible polymeric delivery system suitable for oral administration of bioactive compound, providing coating of the administered compound.

According to one aspect, the present invention provides a plurality of biocompatible polymeric beads having a two-phase core and shell structure. The internal core compartment of each bead comprises a water-in-oil emulsion, further comprising at least one bioactive compound, wherein the compound is either dispersed (for water-insoluble or poorly soluble compounds) or dissolved (for water-soluble compounds) in the discontinuous aqueous phase dispersed within the continuous oil phase of the emulsion, and wherein the internal core compartment surrounded by a polymeric shell compartment comprising biocompatible polymer.

As used herein the term “bioactive compound” refers to any compound having therapeutic or cosmetic activity. Such molecules are exemplified by polylucleotides, proteins, peptides, polysaccharides, hormones, drugs, vitamins, steroids, anti oxidants, anti-inflammatory agents, moisturizers, carotenoids, UV absorbing agents, UV protecting agents and the like.

According to another aspect, the present invention provides a polymeric sustained release delivery system for bioactive compound, the polymeric delivery system comprising a plurality of biocompatible two-phase polymeric beads each bead comprising a core compartment comprising a water-in-oil emulsion surrounded by a polymeric shell compartment comprising a biocompatible polymer, further comprising a bioactive compound dispersed or dissolved in the water phase of the core compartment of the polymeric beads. Thus, the bioactive compound is entrapped within the core water-in-oil emulsion phase of the beads, while the external shell of the beads comprises a biocompatible polymeric matrix, which provides the sustained release characteristics of the system. The core and shell structured as sustained release delivery system is denoted herein as “emulsion beads”. The delivery system preferably comprises a cosmetically effective amount of at least one cosmetic agent or a therapeutic effective amount of at least one therapeutic agent.

The delivery systems of the present invention relate generally to a sustained release polymeric bioactive compound delivery system that is applied directly at a specific body site and preferably permits local release of a water-insoluble or poorly soluble compound or a water-soluble compound for extended periods ranging from a few hours to a few months.

In one specific example, the present biocompatible polymeric delivery system may be used for the controlled release of quinazolinone derivatives having the general formula (1): 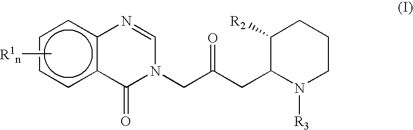
wherein: n=1−2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl;
   and pharmaceutically acceptable salts thereof. Of this group of compounds, halofuginone has been found to be particularly preferred.

In a preferred embodiment, the delivery systems of the present invention are capable of delivering locally a therapeutic dose of a drug, which is higher than the maximum tolerated dose achieved when the drug is administered orally, without inducing the adverse symptoms associated with higher doses of the drug. The sustained release is particularly effective since it eliminates the need for repeated doses throughout the day and avoids the fluctuations in blood levels that are associated with the administration of multiple daily doses.

The biocompatible polymeric bead matrix may be any natural or synthetic biocompatible hydrophilic polymers that are water-soluble prior to polymerization. Preferred natural biocompatible polymers to be used in the present invention are generally polysaccharides or fibrillar proteins. Polysaccharide polymers include for example alginate, dextran, cellulose and cellulose derivatives or chitosan and carrageenan. Additional polysaccharides useful according to the present invention include polyanionic polysaccharides, including dextran sulfate, chondroitin sulfate, heparan sulfate, heparin, keratan sulfate, dermatan sulfate, as well as algal polyglycan sulfates. Polymeric fibrillar proteins include for example gelatin, collagen, elastin, fibrin, and albumin. Preferred synthetic polymers to be used in the present invention are polyacrylic acid polymers, polylactic acid polymers, polycaprolactone polymers, polyglycolic acid and various copolymers thereof. Other polymers that allow the formation of beads by chemical crosslinking or heat-induced solidification may be used in the present invention.

The polymeric drug delivery system of the invention may be implanted to a target site or cavity within a subject as part of an implanted system preferably via a minimally invasive surgical procedure. According to certain embodiments, preferred locations of the implanted delivery system are subcutaneous, or within a body cavity such as a cavity formed following the removal of a tissue during surgery or within any natural body cavity. Such locations may be for example in the brain, kidney capsule, bladder, uterus, vagina, joints, lungs, and peritoneum.

The delivery system of the invention may be applied topically to the desired site for treatment of an intact organism. Suitable sites for topical application of the system include but are not limited to: the skin for dermal administration or transdermal administration; mucosal surfaces including intranasal administration or buccal administration; topical delivery to the lungs by inhalation in the form of aerosols. According to alternative embodiments, the delivery system is administered to the desired location as part of an implanted system or may be positioned directly at the desired site, preferably via a minimally invasive surgical procedure.

According to another aspect the present invention provides a polymeric delivery system for oral administration of a therapeutic agent, the polymeric delivery system comprising biocompatible two-phase polymeric beads, each bead comprising a core compartment comprising a water-in-oil emulsion surrounded by a polymeric shell compartment comprising a biocompatible polymer, further comprising a therapeutic agent dispersed or dissolved in the water phase of the core compartment of the polymeric beads. According to the teaching of the present invention, the therapeutic agent is enclosed within the internal core phase of the emulsion beads, thus the beads serve as an enterocoating preventing the active substance from being released within the stomach as well as in masking undesirable taste, enhancing safety and extending the duration of action by preventing the active substance from being exposed to the acid pH within the stomach. The emulsion beads of the present invention may be used per se or may be further formulated. Oral formulations may be readily prepared by combining the emulsion beads of the present invention with pharmaceutically acceptable diluents or carriers as known in the art. Such carriers enable the delivery systems of the invention to be formulated as capsules, dragees, pills, tablets, gels, liquids, slurries, suspensions, syrups and the like, for oral ingestion by a patient.

The sustained release polymeric delivery system of the present invention exhibits significant advantages over the existing art. Unexpectedly, the beads delivery system permits continuous release of the active compound for prolonged periods and avoids the high initial burst release of the drug as is associated with certain other polymeric delivery systems. Moreover, the polymeric delivery system of the present invention may be structured into an article of a desired shape and size, enabling its application to or at different body locations. The delivery system of the present invention is suitable for incorporating any water-soluble or any water-insoluble or poorly soluble compound while preserving its bioactivity upon exposure to the encapsulation polymer.

While the drug delivery system of the present invention is referred to throughout the specification and claims as “beads”, it is to be understood that this term is intended to be construed in a non-limitative fashion, and do not imply any requisite geometry, specific shape or size of the product. It is noted that the diameter of the beads may vary from several microns to several hundred of microns.

In yet another aspect, the present invention provides methods of preparing the biocompatible polymeric delivery system of the present invention. In one embodiment, a method of preparing core-and-shell-structured polymeric beads comprising the bioactive compound is disclosed. The method comprising: mixing an aqueous solution or dispersion comprising the bioactive compound in an oily phase to form a water-in-oil emulsion; homogenizing the mixture; applying a polymeric shell around small droplets of the emulsion by means of core/shell extension; and solidifying the shell to form two phase core-and-shell-structured polymeric beads.

In yet another aspect, the present invention provides a method of delivering a stable effective concentration of a bioactive compound for extended periods comprising: administrating to a subject in need thereof the biocompatible polymeric delivery system of the present invention comprising the bioactive compound, wherein the delivery system continuously delivers a stable effective concentration of the compound for extended periods. Preferably, the active compound is a cosmetically effective agent or a therapeutic agent. Further preferably, the delivery system continuously delivers the bioactive compound to a specific location in the body. The compound may be water soluble, poorly soluble in water or water insoluble. Of this group of drugs, one example is quinazolinone derivatives having the general formula (I) and salts thereof.

In yet another aspect, the present invention provides a method of treating a disease or disorder in which controlled or sustained release of a therapeutic agent is required, comprising administering to a subject in need thereof the biocompatible polymeric delivery system of the present invention. The therapeutic agent can be selected from the group consisting of, but not limited to, antineoplastic, antimicrobial, antiviral, anticoagulant, antihypertensive, antihistamine, antimalarial, antiepileptic, analgesic, antidepressant, adrenocortical steroid, β-blocker, cardiac glycoside, contraceptive, depressant, hormone, hormone antagonist, immunosuppressant, water-insoluble vitamin, hypoglycemic agent, hyperglycemic agent, mood-altering drug, tranquilizer.

As a non limiting example, the present invention provides a method of treating a disease in which inhibition of angiogenesis, prevention of tumor growth, prevention of smooth muscle cells proliferation or blocking of extracellular matrix deposition (fibrosis) is required, comprising administering to a subject in need the biocompatible polymeric delivery system of the present invention, wherein the delivery system comprising halofuginone entrapped therein, said delivery system continuously delivers a stable therapeutic concentration of halofuginone for extended periods, thereby treating the disease.

These and further embodiments will be apparent from the detailed description and examples that follow.

### BRIEF DESCRIPTION OF THE DRAWINGS

FIG. 1 shows the percentage of halofuginone (HF) released over time from emulsion beads at 37° C., compared to the percentage of halofuginone applied as a solution or as a suspension.

FIG. 2 is an enlargement of FIG. 1, showing the percentage of halofuginone released over time from emulsion beads at 37° C.

FIG. 3 show the release of halofuginone from the emulsion beads, expressed as the concentration of drug (mg/ml) in the external PBS buffer at 37° C.

FIGS. 4-5 are enlargements of FIG. 3.

FIG. 6 shows the percentage of halofuginone released over time from emulsion beads at room temperature, compared to the percentage of halofuginone applied as a soluble drug or suspension drug.

FIG. 7 demonstrate the release of halofuginone from the emulsion beads, expressed as the concentration of drug (mg/ml) in the external PBS buffer at room temperature.

FIG. 8 is an enlargement of FIG. 7.

### DETAILED DESCRIPTION OF THE INVENTION

The present invention relates to biocompatible polymeric beads comprising a bioactive compound, wherein the beads having a two-phase core and shell structure. The present invention further relates to biocompatible polymeric delivery systems that permit controlled release of bioactive compounds, including water-insoluble compounds, compounds poorly soluble in water and water-soluble compounds. The teaching of the present invention is exemplified by the controlled release of a quinazolinone according to Formula (I). Typically the quinazolinone according to Formula (I) is halofuginone or hydrobromide or lactate salts thereof.

The term “water-soluble” compound as used herein refers to a compound that typically has solubility in water in the range of 1 gr/ml to 1 gr/30 ml at room temperature. The term “poorly water-soluble” compound as used herein refers to a compound that typically has solubility in water in the range of 1 gr/30 ml to 1 gr/10,000 ml at room temperature. The term “water-insoluble” compound as used herein refers to a compound that typically has solubility in water of less that 1 gr/10,000 ml at room temperature. The present invention encompasses water-soluble compounds, poorly water-soluble compounds, and water-insoluble compounds.

The polymeric delivery systems of the present invention deliver stable amounts of the active compound for prolonged time periods, preferably within specific location in the body. Variations in the volume of the polymeric matrix provide flexibility in the amount of the bioactive compound released per time period, and the total duration of compound release. Importantly, the present systems eliminate the need for multiple doses of a therapeutic or cosmetic agent given to a subject in need thereof. Furthermore, delivery the systems of the present invention prevent the fluctuations in the compound concentration associated therewith.

According to one embodiment the delivery systems of the present invention are capable of delivering locally a therapeutic dose of a drug which is higher than that achieved by oral administration of the maximum tolerated dose.

According to one embodiment the delivery systems of the present invention are capable of delivering locally a therapeutic dose of halofuginone which is higher than that achieved by oral administration of the maximum tolerated dose. Thus, it is possible to administer halofuginone locally and achieve a therapeutic level higher than that achieved by oral administration of 1 mg/day, which is the maximum tolerated dose of halofuginone with no adverse effects observed in humans when administered orally.

Importantly, the delivery systems of the present invention may avoid or reduce the adverse effects observed with the oral or systemic administration of the drug. Significantly, the use of the sustained release at a target site avoids the need for multiple daily doses of the drug and the resultant fluctuations in serum levels associated therewith.

The present invention can be practiced with a wide variety of therapeutic agents in either the crystalline or the amorphous state. Therapeutic agents with the following utilities can be employed in this invention: antineoplastic, antimicrobial, antiviral, anticoagulant, antihypertensive, antihistamine, antimalarial, antiepileptic, analgesic, antidepressant, adrenocortical steroid, β-blocker, cardiac glycoside, contraceptive, depressant, hormone, hormone antagonist, immunosuppressant, water-insoluble vitamin, hypoglycemic agent, hyperglycemic agent, mood-altering drug, tranquilizer.

Examples of agents that are useful include substances capable of treating or preventing an infection systemically or locally, as for example, antibacterial agents such as penicillin, cephalosporins, bacitracin, tetracycline, doxycycline, quinolines, clindamycin, and metronidazole; antiparasitic agents such as quinacrine, chloroquine and vidarabine; antifungal agents such as nystatin; antiviral agents such as acyclovir, ribarivin and interferons; anti-inflammatory agents such as hydrocortisone and prednisone; analgesic agents such as salicylic acid, acetaminophen, ibuprofen, naproxen, piroxicam, flurbiprofen and morphine; local anesthetics such as lidocaine, bupivacaine, benzocaine, and the like; immunogens (vaccines) for stimulating antibodies against hepatitis, influenza, measles, rubella, tetanus, polio and rabies; peptides such as leuprolide acetate (an LH-RH agonist), nafarelin and ganirelix.

Also useful is a substance or metabolic precursor thereof, which is capable of promoting growth and survival of cells and tissues or augmenting the functioning of cells, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenetic protein, platelet-derived growth factor (PDGF), insulin-derived growth factor (IGF-I, IGF-II), transforming growth factor-alpha (TGF-a), transforming growth factor-.beta. (TGF-β), epidermal growth factor (EGF), fibroblast growth factor (FGF) and interleukin-1 (IL-1); an osteoinductive agent or bone growth promoting substance such as bone chips and demineralized freeze-dried bone material; and antineoplastic agents such as methotrexate, 5-fluoroacil, adriamycin, vinblastine, cisplatin, tumor-specific antibodies conjugated to toxins and tumor necrosis factor.

Other useful substances include hormones such as progesterone, testosterone, and follicle stimulating hormone (FSH) (birth control, fertility-enhancement), insulin metal complexes and somatotropins; antihistamines such as diphenhydramine and chlorpheneramine; cardiovascular agents such as digitalis glycosides, papaverine and streptokinase; anti-ulcer agents such as cimetidine, famotidine and isopropamide iodide; vasodilators such as theophylline, B-adrenergic blocking agents and minoxidil; central nervous system agents such as dopamine; antipsychotic agents such as risperidone, olanzapine; narcotic antagonists such as naltrexone, maloxone and buprenorphine.

Other therapeutic agents are water insoluble anticancer drugs such as carmustine (BCNU), antiviral drugs such as azidothymidine (AZT) and other nucleosides, HIV Protease inhibitors such as saquinavir and retinovir, immune-modulating agents such as cyclosporine, natural and synthetic hormones and hormone regulators such as contraceptives. Other group of therapeutic agents comprises steroidal and non-steroidal anti-inflammatory agents such as hydrocortisone, prednisolone, ketoprofen, celecoxib and ibuprofen. Further applicable therapeutic agents are centrally acting medicines such as antiseptics, antidepressants and sedatives and cardiovascular drugs such as anti-hypertensives and blood lipid lowering agents. Another group of therapeutic agents comprises water insoluble anti-cancer drugs, hormones, analgesics, cardiovascular, antimicrobial or anti-viral agents. The delivery system of the present invention technique is also suitable for immune modulators and drugs that are soluble in dilute acids or bases.

The invention may be applied for bioactive compounds other than therapeutic agents. The bioactive compound is selected from the group consisting of, but not limited to cosmetic agents, polynucleotides, proteins, peptides, polysaccharides, hormones, drugs, vitamins, steroids, anti oxidants, anti-inflammatory agents, moisturizers, carotenoids, UV absorbing agents, UV protecting agents and the like. As disclosed hereinabove, the teaching of the present polymeric delivery system is exemplified by a polymeric delivery system comprises halofuginone as the active drug. Halofuginone is a quinazolinone derivative which was initially used as a coccidiocidal drug but was further discovered to be effective in treating fibrotic diseases, as well as for treatment of restenosis, mesangial cells proliferation, and angiogenesis-dependent diseases (disclosed for example in U.S. Pat. Nos. 6,159,488, 5,998,422, 6,090,814 and 6,028,075). The halofuginone-polymeric beads of the present invention exhibit prolonged release of halofuginone over a period of several months. The formation of oil-in-water or water-in-oil emulsions is a well-known process. Emulsions suitable for generating delivery systems of a therapeutic agent in accordance with the present invention comprise internal core where the drug is either dispersed (for water-insoluble or poorly soluble drugs) or dissolved (for water-soluble drugs) in the discontinuous aqueous phase, an appropriate emulsifier, e.g., a surfactant and a continuous or external phase with limited solubilizing affinity to the dispersed phase. The choice of a suitable emulsifier or a combination of emulsifiers can readily be made by those skilled in the art. Surfactants which can be used for this purpose may be selected from the following groups:

Reaction products of natural or hydrogenated vegetable oils, and ethylene glycol; i.e., polyoxyethylene glycolated natural or hydrogenated vegetable oils: for example polyoxyethylene glycolated natural or hydrogenated castor oils. Surfactants commercialized under the trade names Cremophor RH-40, Cremophor RH60, Cremophor EL, Nikkol HCO-40 and Nikkol HCo-60 may be used in the composition according to the present invention. Cremophor RH40 and Cremophor El are preferred.

Polyoxyethylene sorbitan fatty acid esters: e.g., mono- and tri-lauryl, palmityl, stearyl and oleyl esters; e.g. products of the trade name “Tween,” which includes polyoxyethylene sorbitan mono-laurate (Tween), polyoxyethylene sorbitan mono-palmitate (Tween 40), polyoxyethylene sorbitan mono-oleate (Tween 80), etc. depending on the kind of fatty acid. Tween 20 and Tween 40 can be used preferably in the composition according to the present invention.

Polyoxyethylene fatty acid esters: for example, polyoxyethylene stearic acid esters of the type known and commercially available tinder the trade name Myrj as well as polyoxyethylene fatty acid esters known and commercially available under the trade name “Cetiol HE.” Polyoxyethylene-polyoxypropylene co-polymers: e.g. of the type known and commercially available tinder the trade names “Pluronic” and “Emkalyx.”

Polyoxyethylene-polyoxypropylene block co-polyiners: e.g. of the type known and commercially available under the trade name “Poloxamer.” Dioctylsuccinate, dioctylsodiumsulfosuccinate, di-[2-ethylhexyl]-succinate or sodium lauryl sulfate.

Phospholipids, in particular lecithins: especially, soybean lecithin.

Surfactants such as non-ionic polyoxyethylene fatty acid derivatives, in particular, polyoxyethylene sorbitan fatty acid esters (spans) such as sorbitan sesquiolate are preferred for use as emulsifiers.

Emulsification is usually performed by applying mechanical force to break down the internal phase liquid into small globules, in the range of 10 mm, to several micrometers in diameter. Such mechanical force can be applied by mechanical stirring, ultrasonic probes, or by passing the emulsion components through narrow space, as in the case of colloidal mills, or through narrow tubes, nozzles, valves or orifices.

The biocompatible polymeric bead matrix may be any natural or synthetic biocompatible hydrophilic polymers. Hydrophilic polymers including alginates and derivatives thereof can be obtained from various commercial, natural or synthetic sources well known in the art. As used herein, the term hydrophilic polymer refers to water-soluble polymers or polymers having affinity for absorbing water. Hydrophilic polymers are well known to one skilled in the art. These include but are not limited to polyanions, including anionic polysaccharides such as alginate, carboxymethyl amylose, polyacrylic acid salts, polymethacrylic acid salts, ethylene maleic anhydride copolymer (half ester), carboxymethyl cellulose, dextran sulfate, heparin, carboxymethyl dextran, carboxy cellulose, 2,3-dicarboxycellulose, tricarboxycellulose, carboxy gum arabic, carboxy carrageenan, pectin, carboxy pectin, carboxy tragacanth gum, carboxy xanthan gum, pentosan polysulfate, carboxy starch, carboxymethyl chitin/chitosan, curdlan, inositol hexasulfate, β-cyclodextrin sulfate, hyaluronic acid, chondroitin-6-sulfate, dermatan sulfate, heparin sulfate, carboxymethyl starch, carrageenan, polygalacturonate, carboxy guar gum, polyphosphate, polyaldehydo-carbonic acid, poly-1-hydroxy-1-sulfonate-propen-2, copolystyrene maleic acid, agarose, mesoglycan, sulfopropylated polyvinyl alcohols, cellulose sulfate, protamine sulfate, phospho guar gum, polyglutamic acid, polyaspartic acid, polyamino acids, derivatives or combinations thereof. One skilled in the art will appreciate other various hydrophilic polymers that are within the scope of the present invention.

According to one embodiment, the delivery system of the invention is implanted directly to the site of action, preferably via a minimally invasive surgical procedure. For example, the system of the invention may be implanted subcutaneously, using procedures known to those skilled in the art. The beads may be administered subcutaneously by injection using appropriate syringes. In another embodiment, the system of the invention may be implanted in any body cavity such as for example via laparoscopy, or endoscopy. In another embodiment, the system of the invention may be implanted in any body cavity such as for example in the uterus, brain, kidney capsule, bladder, vagina, joints, lungs, and peritoneum. In yet another embodiment, the system of the invention may be implanted in a cavity formed during a surgical procedure, such as but not limited to surgery for the removal of a malignant tissue.

According to another aspect the present invention provides a polymeric delivery system for oral administration of a therapeutic agent, the polymeric delivery system comprising biocompatible two-phase polymeric beads each bead comprising a core compartment comprising a water-in-oil emulsion surrounded by a polymeric shell compartment comprising a biocompatible polymer, further comprising a therapeutic agent dispersed or dissolved in the water phase of the core compartment of the polymeric beads. The polymeric beads, providing coating of the active ingredient, prevents the undesirable taste of the drug ingredient from coming through, making the drug preparation more palatable; enhances the safety in consuming the drug and extends the duration of the drug action.

The polymeric beads of the present invention can be further formulated for oral administration by combining the emulsion beads of the present invention with pharmaceutically acceptable diluents or carriers as known in the art. Such carriers enable the delivery systems of the invention to be formulated as capsules, dragees, pills, tablets, gels, liquids, slurries, suspensions, syrups and the like, for oral ingestion by a patient.

Solid forms for oral administration include capsules, tablets, pills, powders and granules. In such solid forms, the emulsion beads can be admixed with at least one inert diluent, such as sucrose, lactose or starch. Such oral forms can also comprise, additional substances other than inert diluent. In the case of capsules, tablets and pills, the formulation may also comprise buffering agents. Tablets and pills can additionally be prepared with an enteric coating.

Liquid forms for oral administration include pharmaceutically acceptable emulsions, solutions, suspensions, syrups and elixirs, containing inert diluents commonly used in the pharmaceutical art. Besides inert diluents, such compositions can also include adjuvants, such as wetting agents, emulsifying and suspending agents, and sweeteners.

In another embodiment, the delivery system of the invention may be applied topically in a target site of an intact organism. Preferred targets for topical application of the system are for example: the skin using transdermal administration, intranasal administration and topical delivery to the lungs as aerosols. For transdermal administration it is desirable that the beads will be dispersed with oils to provide an oily suspension, emulsion, cream or gel.

One skilled in the art will be able to ascertain effective dosages of the bioactive compounds to be administered via the delivery system of the present invention by administration and observing the desired therapeutic effect. The dosage of the sustained-release preparation is the amount necessary to achieve the effective concentration of the compound in vivo, for a given period of time. The dosage and the preferred administration frequency of the sustained-release preparations vary with the desired mode of action, desired duration of the release, the target disease, desired administration frequency, the subject animal species and other factors. In one specific example, the drug to be administered is halofuginone. Preferably, the total amount of halofuginone to be administered via the delivery system of the present invention may be between about 0.1 mg/day and about 10 mg/day.

As disclosed herein below, a specific example of the delivery system according to the present invention comprises halofuginone as the therapeutic agent. In this particular case, the delivery system of the invention can be used in treating fibrotic diseases, restenosis, glomerulosclerosis, cancer and other angiogenesis-dependent diseases. The delivery system comprising halofuginone may be preferably used in treating diseases in which inhibition of tumor progression by cell cycle arrest, cell invasiveness or inhibiting angiogenesis is required, or in treating diseases in which blocking of extracellular matrix deposition is required. Clinical conditions and disorders associated with primary or secondary fibrosis, such as systemic sclerosis, graft-versus-host disease (GVHD), pulmonary and hepatic fibrosis and a large variety of autoimmune disorders are distinguished by excessive production of connective tissue, which results in the destruction of normal tissue architecture and function. These diseases can be interpreted in terms of perturbations in cellular functions, a major manifestation of which is excessive collagen deposition.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Experimental Procedures

Emulsion Beads:

Both Emulsion and Suspension beads experiments were conducted with micronized halofuginone (HF HBr), batch H001. A water-in-oil emulsion was prepared, in which the 20% wt internal phase contained 50 mg HF HBr/ml and the oil was sunflower oil. The emulsion was prepared by adding the aqueous HF solution (contains 50 mg/ml HF HBr, 0.3% wt Tween 80) into the oil which contains 2.7% wt Span 80, and homogenizing by an Ultra turrax homogenizer (2 min at 13,000 rpm and 10 min at 16,000 rpm). Beads were formed by a core-shell double nozzle Innotek (500 and 400 microns), flow rate of the core material 90 (instrument scale) pressure (shell) 0.6 Atm. The shell solution was 2.5% sodium alginate (FMC LF10/60) and 2.5% silica in aqueous solution (Theoretical Shel/core weight ratio 15:1 by volume).

The crosslinking solution was 100 mM CaCl₂, or 100 mM NaCl+100 mM CaCl₂. The purpose of the crosslinking solution is to provide the insoluble polymeric coating. The properties of the polymeric shell depend on various parameters, such as NaCl/CaCl₂ ratio.

For the release experiments, 300 mg beads were suspended in 1 ml PBS buffer, and put into a dialysis tube, while the tube immersed in 10 ml PBS. Therefore, the maximal concentration of HF, which can be released is 0.36 mg/ml. For all experiments, the concentration measurements were performed by a UV-spectrophotometer, while using a calibration curve of HF PBS solution. The dialysis was performed while shaking, at 37° C. at 5 strokes/min. The external buffer was completely replaced after each measurement.

Release Experiments—Controls

Suspended drug: 100 mg HF/ml PBS. 1 ml of the suspension was put in the dialysis tube. The external solution was 10 ml PBS. Maximal drug concentration, which can be released in these conditions, is 9.09 mg/ml.

Soluble drug: 0.2 mg/ml solution of HF in PBS. 1 ml of the solution was put in the dialysis tube. The external solution was 10 ml PBS. Maximal drug concentration, which can be released in these conditions, is 0.018 mg/ml.

### Example 1

### Extended Release of Halofuginone (HF) Using Alginate Beads

In the first set of experiments, the release of HF from the Emulsion beads was conducted in 37° C. The release pattern is presented both as the percentage of drug released of the total expected drug release and as the actual measured concentration.

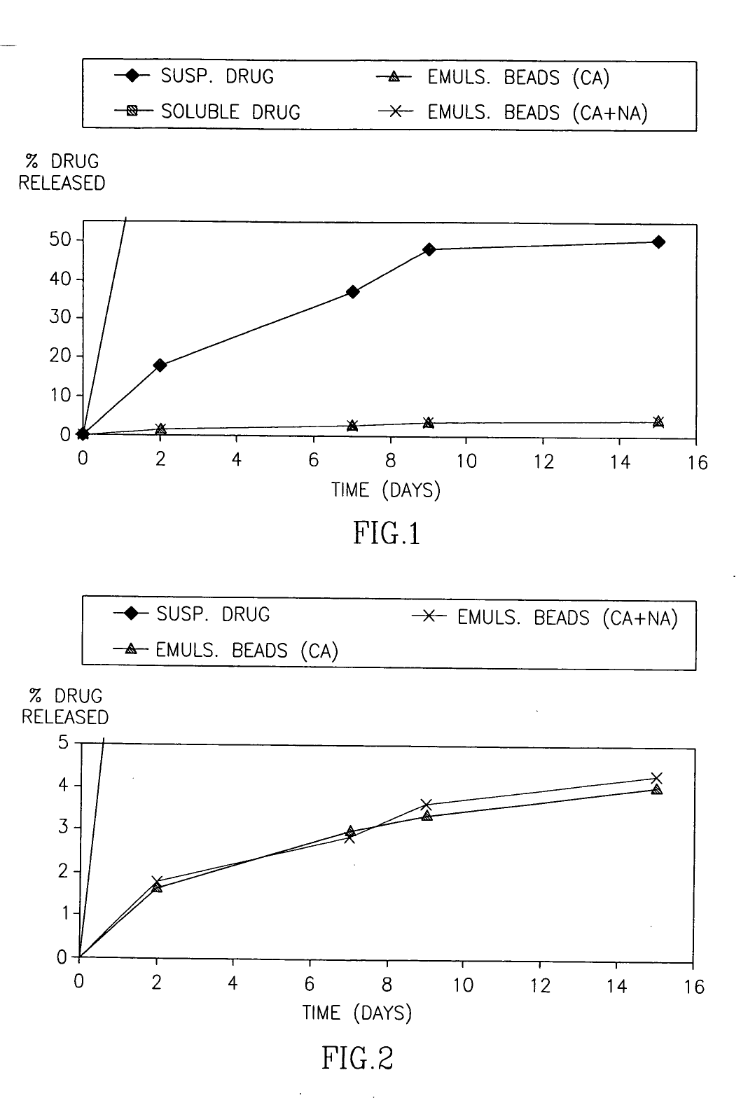

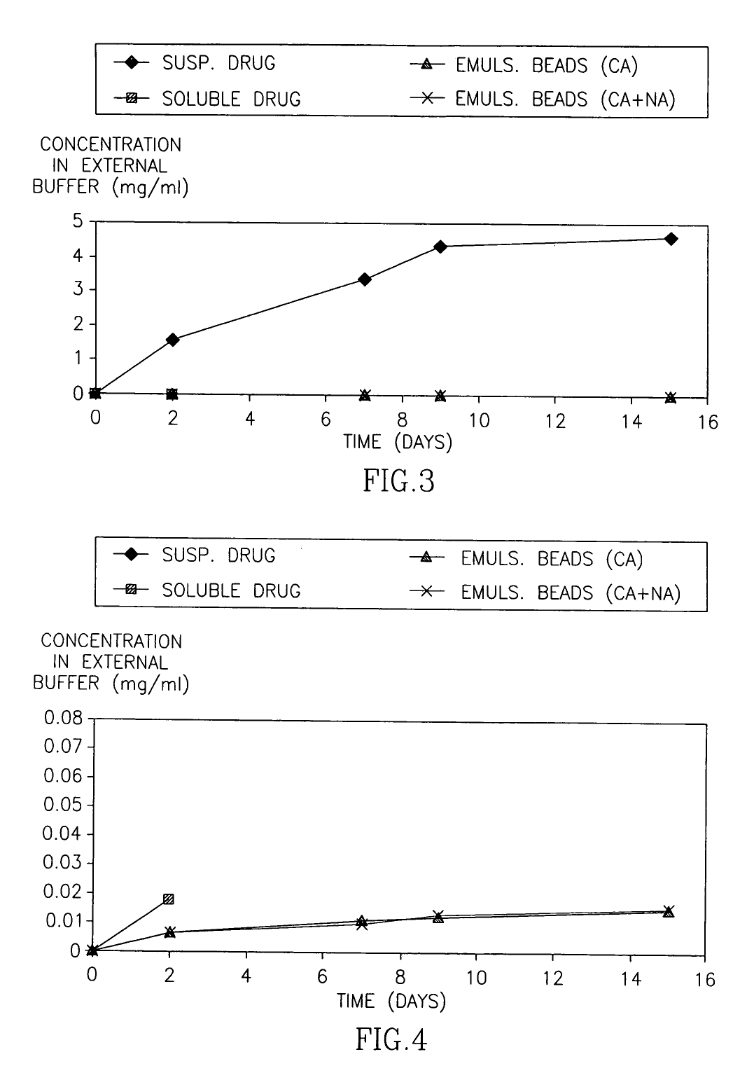

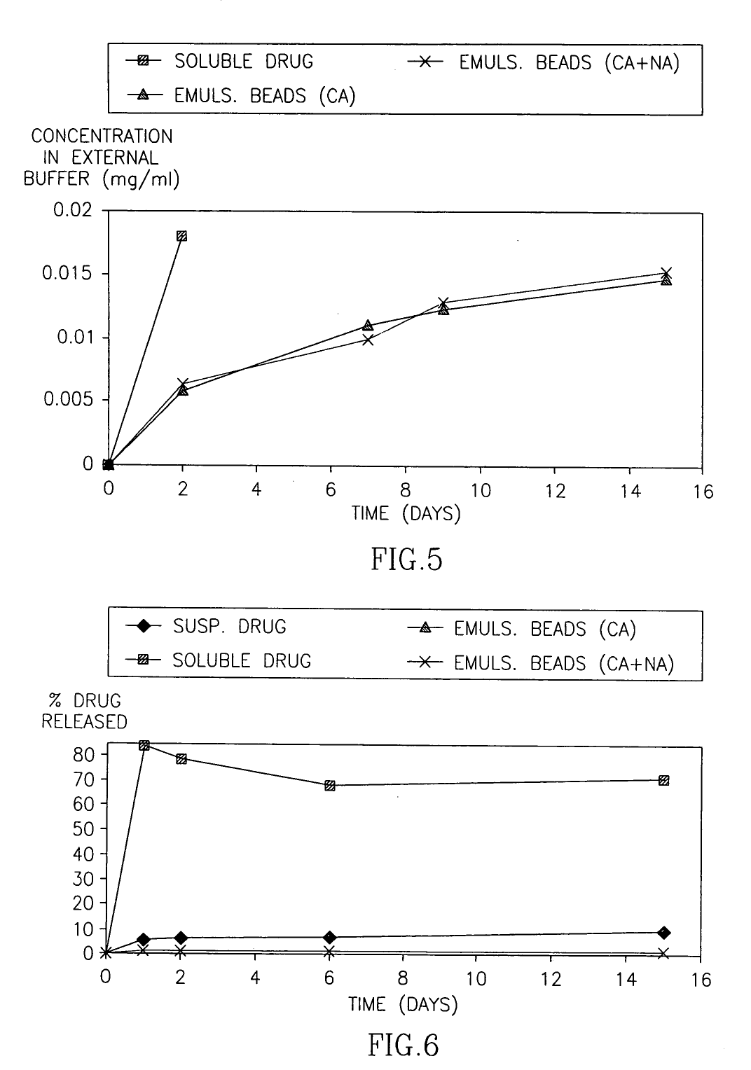

FIG. 1 demonstrates the cumulative percentage of HF released over time from emulsion beads, compared to halofuginone applied in a solution or suspension. FIG. 2 is an enlargement of FIG. 1 demonstrating the consistent drug release from the Emulsion beads over time. FIGS. 3-5 demonstrate the release of HF from the Emulsion beads, expressed as the cumulative concentration of drug (mg/ml) in the external PBS buffer.

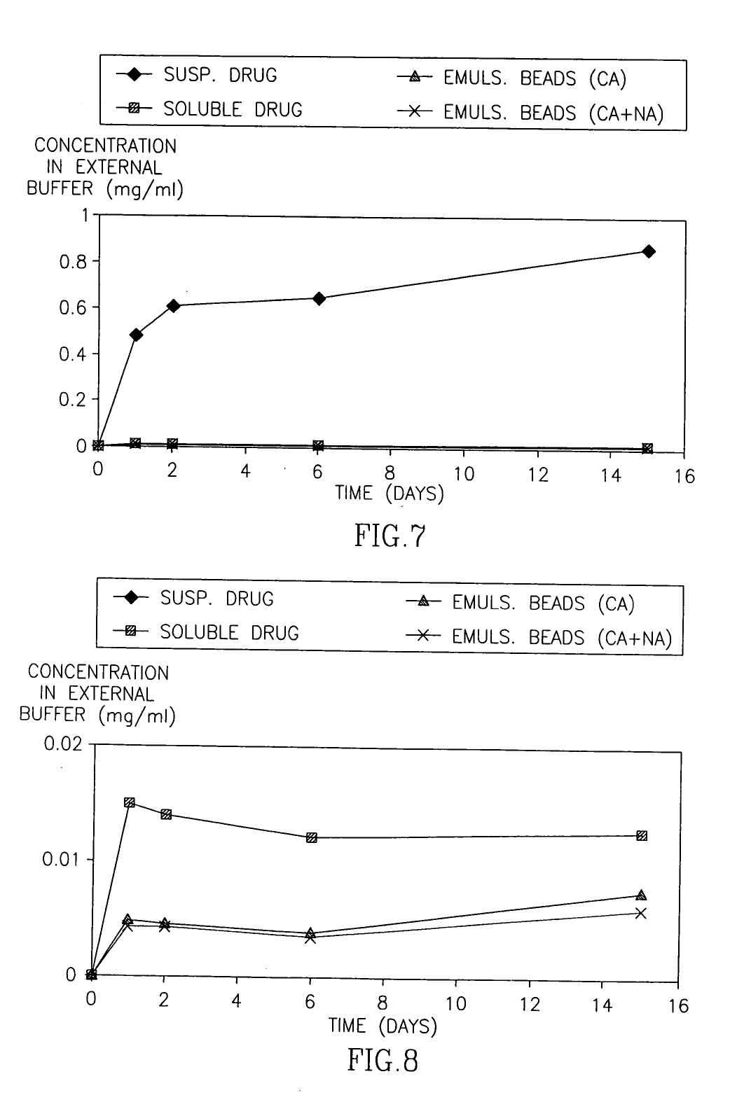

In the second set of experiments, the release of HF from the Emulsion beads was conducted at room temperature. FIG. 6 demonstrates the cumulative percentage of HF released over time from the emulsion beads. FIGS. 7-8 demonstrate the release of HF from the emulsion beads expressed as the cumulative concentration of drug (mg/ml) in the external PBS buffer.

As demonstrated in FIGS. 1-8, it is possible to use the Emulsion beads as a delivery system for the HF. Furthermore, the drug release from the beads is much slower as compared to HF solution or suspension.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1. A plurality of biocompatible two-phase polymeric beads, each bead comprising a core compartment comprising a water-in-oil emulsion surrounded by a polymeric shell compartment comprising a biocompatible polymer, the core compartment further comprising at least one bioactive agent.

2. The polymeric beads of claim 1 wherein the bioactive compound is selected from a water-soluble compound, a compound poorly soluble in water and a water insoluble compound.

3. The polymeric beads of claim 2 wherein the bioactive compound is a cosmetic agent selected from the group consisting of anti oxidants, anti-inflammatory agents, moisturizers, vitamins, carotenoids, UV absorbing agents and UV protecting agents.

4. The polymeric beads of claim 2 wherein the bioactive compound is a therapeutic agent selected from the group consisting of antineoplastic, antimicrobial, antiviral, anticoagulant, antihypertensive, antihistamine, antimalarial, antidepressant, and antiepileptic drug; hormone, hormone antagonist, water-insoluble vitamin, cardiac glycoside, tranquilizer, adrenocortical steroid, β-blocker, contraceptive, depressant, immunosuppressant, analgesic, hypoglycemic agent, hyperglycemic agent, and mood-altering drug.

5. The polymeric beads of claim 1 wherein the biocompatible polymer is a natural or synthetic hydrophilic polymer, a polysaccharide or a protein.

6. A polymeric delivery system for sustained release of a bioactive compound, the polymeric delivery system comprising biocompatible two-phase polymeric beads each bead comprising a core compartment comprising a water-in-oil emulsion surrounded by a polymeric shell compartment comprising a biocompatible polymer, the core compartment further comprising at least one bioactive agent.

7. The delivery system of claim 6, wherein the polymeric beads are dispersed within an oil-based formulation to provide an oily suspension, emulsion, cream or gel.

8. The delivery system of claim 6, wherein the at least one bioactive compound is a water-soluble compound, a compound poorly soluble in water or a water insoluble compound and is dispersed or dissolved in the water phase of the core compartment of the polymeric beads.

9. The delivery system of claim 8 wherein the bioactive agent is a cosmetic agent selected from the group consisting of anti oxidants, anti-inflammatory agents, moisturizers, vitamins, carotenoids, UV absorbing agents and UV protecting agents.

10. The delivery system of claim 8 wherein the bioactive agent is therapeutic agent selected from the group consisting of antineoplastic, antimicrobial, antiviral, anticoagulant, antihypertensive, antihistamine, antimalarial, antidepressant, or antiepileptic drug; hormone, hormone antagonist, water-insoluble vitamin, cardiac glycoside, tranquilizer, adrenocortical steroid, p-blocker, contraceptive, depressant, immunosuppressant, analgesic, hypoglycemic agent, hyperglycemic agent, and mood-altering drug.

11. The delivery system of claim 8, wherein the biocompatible polymer is a natural or synthetic hydrophilic polymer, a polysaccharide or a protein.

12. The delivery system of claim 8, wherein the bioactive compound is released at a therapeutically effective concentration for a time period ranging from a several hours to a several months.

13. The delivery system of claim 12 wherein the route of administration is selected from implantation, subcutaneous injection and deposition within a body cavity.

14. The delivery system of claim 8 as a formulation for oral administration, wherein the bioactive compound is a therapeutic agent, and is dispersed or dissolved in the water phase of the water-in-oil emulsion in the core compartment.

15. The polymeric delivery system of claim 14 wherein the formulation is a capsule, a dragee, a pill, a tablet, a gel, a liquid, a slurry, a suspensions, or a syrup, and wherein the beads mask the unpleasant taste of the therapeutic agent or act as an enterocoating.

16. A method of preparing the biocompatible polymeric beads, comprising:
(a) mixing an aqueous solution or suspension of the bioactive compound in an oily phase to form a water-in-oil emulsion, in the presence of at least one surface active agent;
(b) homogenizing the mixture of step (a);
(c) applying a polymeric shell around small droplets of the emulsion by means of core/shell extrusion; and
(d) solidifying the shell to form two phase core-and-shell-structured polymeric beads.

17. A method of delivering a stable therapeutic concentration of a therapeutic agent, comprising: administering to a subject in need thereof the biocompatible polymeric delivery system according to claim 6, wherein said delivery system continuously delivers a stable therapeutic concentration of the agent for a period of time ranging from hours to months.

18. The method of claim 17, wherein the therapeutic agent is selected from the group consisting of antineoplastic, antimicrobial, antiviral, anticoagulant, antihypertensive, antihistamine, antimalarial, antidepressant, or antiepileptic drug;
hormone, hormone antagonist, water-insoluble vitamin, cardiac glycoside, tranquilizer, adrenocortical steroid, β-blocker, contraceptive, depressant, immunosuppressant, analgesic, hypoglycemic agent, hyperglycemic agent, and mood-altering drug.

19. A method of treating or inhibiting a disease or disorder in which a sustained release of a therapeutic agent is required comprising administering to a subject in need thereof a biocompatible polymeric delivery system according to claim 6, the delivery system continuously delivering a stable therapeutic concentration of the agent for a period of time ranging from hours to months, thereby treating or preventing the disease or disorder.

20. The method of claim 19 wherein the therapeutic agent is selected from the group consisting of antineoplastic, antimicrobial, antiviral, anticoagulant, antihypertensive, antihistamine, antimalarial, antidepressant, or antiepileptic drug; hormone, hormone antagonist, water-insoluble vitamin, cardiac glycoside, tranquilizer, adrenocortical steroid, β-blocker, contraceptive, depressant, immunosuppressant, analgesic, hypoglycemic agent, hyperglycemic agent, and mood-altering drug.

21. An implant comprising the polymeric delivery system of claim 6.
